(19) United States
(12) Patent Application Publication
(10) Pub. No.: US 2023/0212651 A1
(43) Pub. Date: Jul. 6, 2023

(54) Detection of Target Oligonucleotides

(71) Applicant: TTP PLC, Melbourn, Royston Hertfordshire (GB)

(72) Inventors: Rebecca Louise Howard, Melbourn (GB); Kathrin Herbst, Melbourn (GB); Giles Hugo William Sanders, Melbourn (GB)

(21) Appl. No.: 17/629,864

(22) PCT Filed: Jul. 24, 2020
(86) PCT No.: PCT/GB2020/051782

§ 371 Date: Jan. 25, 2022

(30) Foreign Application Priority Data
Jul. 25, 2019 (GB) 1910635.0

(51) Int. Cl. C12Q 1/6816; C12Q 1/6806; C12Q 1/686

(52) U.S. Cl. CPC C12Q 1/6816; C12Q 1/6806; C12Q 1/686

(57) ABSTRACT

There is provided a method of detecting the presence of a nucleic acid target sequence in which two oligonucleotides are used to forma three-way junction with the target sequence to allow detection of the target sequence. Alternatively, three oligonucleotides can be used to form a four-way junction with the target sequence to allow detection of the target sequence.

This application incorporates by reference the contents of a 1,395 byte text file named “SubstituteSequenceListing000487.00205” created on Dec. 8, 2022, which is the sequence listing for this application.

### FIELD OF THE INVENTION

The present invention relates to a method of detecting a specific target oligonucleotide sequence that involves the use of a DNA/RNA junction system. Where the target oligonucleotide is RNA, the method allows for RNA analysis without any enzymes to convert RNA to DNA.

### BACKGROUND TO THE INVENTION

Ribonucleic acids (RNAs) are of interest in life sciences and diagnostics for a number of reasons—they are essential in determining levels at which genes are expressed and encode the genomes of many pathogenic retroviruses. While the polymerase chain reaction (PCR) is commonly used for the detection of DNA, this is not compatible with RNA, which must first be transcribed into cDNA in what is known as a reverse transcription step. This step requires use of an enzyme family known as a reverse transcriptase. Initially reverse transcription was performed independently of DNA amplification, but technology has progressed to the extent that both reverse transcription and quantitative PCR (qPCR) can be performed in a single reaction. Amplification reaction mixes able to perform both a reverse transcription and quantitative PCR (RT-qPCR mixes) are currently significantly more expensive than qPCR mixes alone, since enzymes are the most expensive component of the reaction mix. One invention designed to address the need for multiple enzymes is the Tth enzyme, which is able to perform both reverse transcription and amplification reactions. While the Tth enzyme is compatible with both reverse transcription and qPCR, its purchase cost is over ten-fold higher than that of a standard Taq polymerase (Sigma-Aldrich) thus while this discovery does reduce the need for multiple enzymes, it does not address the issue of cost. The Tth enzyme is also reputed to have a lower overall sensitivity and specificity than a common two-enzyme system.

Whilst the vast majority of detection methods involve the use of reverse transcription to convert RNA to cDNA, additional methods have been developed that permit detection of RNA without amplifying it directly. One example is a method which involves detecting RNA, then amplifying the resultant signal rather than amplifying the nucleic acid itself. This is generally done by some form of cascade reaction. Another solution is to use the quenching properties of graphene oxide with competitive hybridisation for direct RNA detection. Both are comparatively slower than PCR.

As is known to those skilled in the art, following the enzymes, typically the second most expensive component of a qPCR reaction, where used, is the detection probe. While for simple amplification reactions it is possible to use an intercalating dye for detection, it is more common when multiplexing to incorporate a target-specific fluorescent probe.

There have been many innovations aimed at incorporating universal fluorescent detection systems. Three such examples are amplifluor, KASP amplification, and a universally synthesised molecular probe element to which a target-specific element may be added.

Junction systems such as the four-way Holliday junction occur naturally in nature, for example, they are formed during DNA replication and repair events. RNA is also known to be involved in forming comparable structures. As a result, there have been attempts to use such junction systems for nucleic acid detection. For RNA detection specifically, a three-way junction system has been described combined with rolling circle amplification to eliminate the need for reverse transcription (e.g. see Taku Murakami et al., Nucleic Acids Research, 2012, 40(3): e22). However, this method still has drawbacks, including the requirement for an additional nicking enzyme (nickase).

### SUMMARY OF THE INVENTION

The present invention relates to the detection of a nucleic acid target sequence through the formation of a junction structure that only forms in the presence of the target sequence and facilitates detection of a double-stranded nucleic acid.

In a first embodiment, the present invention provides a method of detecting the presence of a nucleic acid target sequence, the method comprising the steps of:
a) adding a first, a second and a third oligonucleotide to a sample comprising nucleic acid sequences, wherein:
   i. the first oligonucleotide comprises a first portion and a second portion, the first portion being complementary to a first portion of the second oligonucleotide and the second portion being complementary to a first portion of the target sequence;
   ii. the second oligonucleotide comprises a first portion and a second portion, the first portion being complementary to the first portion of the first oligonucleotide and the second portion being complementary to a second portion of the target sequence;
b) adding a polymerase to the sample, wherein when the target sequence is present in the sample:
   i. the polymerase initiates nucleic acid synthesis from the first oligonucleotide using the second oligonucleotide as a template strand to generate an extended first oligonucleotide;
   ii. the third oligonucleotide comprises a portion that is complementary and hybridises to the newly synthesised portion of the extended first oligonucleotide; and
   iii. the polymerase initiates nucleic acid synthesis from the third oligonucleotide using the extended first oligonucleotide as a template strand to generate a double stranded nucleic acid;
c) detecting the double stranded nucleic acid.

In the method defined above, when the target sequence is present in the sample, the target sequence, the first oligonucleotide and the second oligonucleotide hybridise as a result of the complementary portions of their sequences. The hybridisation of the target sequence, the first oligonucleotide and the second oligonucleotide forms a complex, in particular, a three-way junction. When the target sequence is not present in the sample, the first oligonucleotide and the second oligonucleotide do not hybridise.

The term ‘complementary’ means that the relevant portions of the oligonucleotides have bases which are complementary and which can form hydrogen bonded base pairs. Guanine is the complementary base of cytosine, and adenine is the complementary base of thymine in DNA and of uracil in RNA. The hydrogen bonding of the complementary portions allows them to hybridise to each other under appropriate conditions, e.g. when the target sequence is present to allow the first and second oligonucleotides to form a complex with the target sequence. The bases in complementary portions of the oligonucleotides are at least 95% complementary, more preferably at least 98% complementary, and in some embodiments, 100% complementary.

The first, second and third oligonucleotides, described hereinafter as ‘the oligonucleotides’ may be formed from DNA, peptide nucleic acids (PNA), locked nucleic acids (LNA) or any combination thereof. Preferably, the oligonucleotides are formed from DNA. In some embodiments, the first oligonucleotide is formed from DNA, the second oligonucleotide is formed from DNA, PNA, LNA or any combination thereof, and the third oligonucleotide is formed from DNA.

The oligonucleotides may be any length of nucleotides (nt) which allow them to effectively hybridise to their respective complementary sequences.

### First Oligonucleotide

The first oligonucleotide may be 10-80 nt in length. In some embodiments, the first oligonucleotide is 15-70 nt. In various embodiments, the first oligonucleotide is 20-60 nt. In a number of embodiments, the first oligonucleotide is 25-55 nt. In certain embodiments, the first oligonucleotide is 30-50 nt. In particular embodiments, the first oligonucleotide is 35-45 nt.

The first oligonucleotide comprises a first portion and a second portion, the first portion being complementary to a first portion of the second oligonucleotide and the second portion being complementary to a first portion of the target sequence.

The first portion of the first oligonucleotide may be 5-40 nt in length. In some embodiments, the first portion of the first oligonucleotide is 10-35 nt. In various embodiments, the first portion of the first oligonucleotide is 15-30 nt. In a number of embodiments, the first portion of the first oligonucleotide is 15-25 nt.

The second portion of the first oligonucleotide may be 5-40 nt in length. In some embodiments, the second portion of the first oligonucleotide is 10-35 nt. In various embodiments, the second portion of the first oligonucleotide is 15-30 nt. In a number of embodiments, the second portion of the first oligonucleotide is 15-25 nt.

In some embodiments, the first portion of the first oligonucleotide is positioned on the 3′ side of the second portion. In various embodiments, the first portion is positioned at the 3′ end of the first oligonucleotide.

In a number of embodiments, the first portion and second portion of the first oligonucleotide are separated by 0-10 nucleotides. In particular embodiments, the first portion and second portion are separated by 0-5 nucleotides. In certain embodiments, the first portion and second portion are separated by 0-2 nucleotides. In some embodiments, the first portion and second portion are separated by 0 nucleotides, i.e. they are contiguous.

### Second Oligonucleotide

The second oligonucleotide may be 50-250 nt in length. In some embodiments, the second oligonucleotide is 70-200 nt. In various embodiments, the second oligonucleotide is 90-180 nt. In a number of embodiments, the second oligonucleotide is 100-170 nt. In certain embodiments, the second oligonucleotide is 110-160 nt. In particular embodiments, the second oligonucleotide is 120-150 nt.

The second oligonucleotide comprises a first portion and a second portion, the first portion being complementary to the first portion of the first oligonucleotide and the second portion being complementary to a second portion of the target sequence.

The second oligonucleotide comprises a third portion that is homologous to the third oligonucleotide. When the first oligonucleotide is extended using the second oligonucleotide as the template strand, the complement to the third oligonucleotide is synthesised.

The first portion of the second oligonucleotide may be 5-40 nt in length. In some embodiments, the first portion of the second oligonucleotide is 10-35 nt. In various embodiments, the first portion of the second oligonucleotide is 15-30 nt. In a number of embodiments, the first portion of the second oligonucleotide is 15-25 nt.

The second portion of the second oligonucleotide may be 5-40 nt in length. In some embodiments, the second portion of the second oligonucleotide is 10-35 nt. In various embodiments, the second portion of the second oligonucleotide is 15-30 nt. In a number of embodiments, the second portion of the second oligonucleotide is 15-25 nt.

In some embodiments, the first portion of the second oligonucleotide is positioned on the 5′ side of the second portion. In various embodiments, the second portion is the closest portion to the 3′ end of the second oligonucleotide. In certain embodiments, the second portion is positioned at the 3′ end of the second oligonucleotide.

In a number of embodiments, the first portion and second portion of the second oligonucleotide are separated by 0-10 nucleotides. In particular embodiments, the first portion and second portion are separated by 0-5 nucleotides. In certain embodiments, the first portion and second portion are separated by 0-2 nucleotides. In some embodiments, the first portion and second portion are separated by 0 nucleotides, i.e. they are contiguous.

The second oligonucleotide is used as a template strand when the first oligonucleotide is extended by the polymerase. The portion of the second oligonucleotide that is used as a template strand may be 50-200 nt. In some embodiments, this portion is 60-140 nt. In various embodiments, this portion is 70-120 nt. In a number of embodiments, this portion is 80-100 nt.

In various embodiments, the portion of the second oligonucleotide that is used as a template strand is positioned on the 5′ side of the first portion. This portion may comprise the third portion that is homologous to the third oligonucleotide.

### Third Oligonucleotide

The third oligonucleotide may be 10-50 nt in length. In a number of embodiments, the third oligonucleotide is 10-45 nt. In certain embodiments, the third oligonucleotide is 15-40 nt. In particular embodiments, the third oligonucleotide is 20-35 nt.

The third oligonucleotide comprises a portion that is complementary and hybridises to the newly synthesised portion of the extended first oligonucleotide. Put another way, the third oligonucleotide comprises a portion that is homologous to a third portion of the second oligonucleotide (which is used as a template strand when the first oligonucleotide is extended).

The term ‘homologous’ means that the relevant portions of the oligonucleotides which are homologous have base sequences which are substantially the same. The base sequences in homologous portions of the oligonucleotides have at least 95% sequence identity (i.e. 95% of the bases are the same). More preferably, the base sequences in homologous portions have at least 98% sequence identity. In some embodiments, the base sequences in homologous portions have 100% sequence identity, i.e. the sequences are the same.

The oligonucleotides may be synthesised using any method known to one skilled in the art. The oligonucleotides may be composed of entirely canonical nucleobases. Alternatively, the oligonucleotides may comprise canonical and non-canonical nucleobases. The oligonucleotides may be unmodified. Alternatively, at least one of the oligonucleotides may be modified. The oligonucleotides may be modified by any technique known to one skilled in the art. Such techniques include modifying the DNA backbone, e.g. bridged nucleic acids, methylphosphonate backbone, phosphodiester (PO) backbone, phosphorothioate (PS) backbone and phosphorodithioate (PdiS) backbone. Such techniques also include modifying the bases, e.g. 2-aminopurine, 2,6-diaminopurine (2-amino-dA), 5-bromo-deoxyuridine, deoxyuridine, inverted dT, inverted dideoxy-T, dideoxycytidine, 5-methyl deoxycytidine, deoxyinosine, 5-hydroxybutynl-2′-deoxyuridine, 8-aza-7-deazaguanosine, locked nucleic acids (LNAs), peptide nucleic acids (PNAs), 5-nitroindole, 2′-O-methyl RNA bases, hydroxymethyl dC, iso-dC, iso-dG, fluorescently-labelled bases and 2′-O-methoxy-ethyl bases (2′-MOE).

The target sequence may be any nucleic acid. The target sequence may be DNA or RNA. Preferably, the target sequence is RNA. The target sequence may be a sequence from a pathogen (such that the complex may be used to detect the presence of a pathogen) or a microbe, or may be the sequence of a particular human, animal or plant allele, such that the genotype or phenotype of an individual human, animal or plant may be determined. The target sequence may be part of a genome, a gene, a gene fragment, a plasmid, an oligonucleotide, an artificial gene or an artificial gene fragment.

The polymerase may be any nucleic acid polymerase which is suitable for initiating nucleic acid synthesis from the first oligonucleotide and third oligonucleotide. The polymerase is preferably a DNA polymerase. The polymerase catalyses the synthesis of nucleic acid. The polymerase may be thermostable. The polymerase may be selected from Taq DNA polymerase, Pfu DNA polymerase, KOD DNA polymerase, Bst DNA polymerase, Bst 2.0 DNA polymerase, Bst 3.0 DNA polymerase, Phi29 DNA polymerase, Bsu DNA polymerase, Tth polymerase, Pwo DNA polymerase, T7 DNA polymerase, Q5 polymerase, Vent DNA polymerase, Klenow fragment of DNA polymerase I, Φ29 polymerase, GspSSD DNA Polymerase I, GspSSD2.0 DNA Polymerase I, GspSSD3.0 DNA Polymerase I, Tin(exo-) LF DNA Polymerase and Sequenase, and functional fragments thereof. Preferably, the polymerase is a thermostable polymerase suitable for use in routine PCR. Preferably, the polymerase is a modified or unmodified Taq DNA polymerase.

As indicated above, the first oligonucleotide, the second oligonucleotide and the target sequence hybridise to each other at their respective complementary regions to form a three strand structure. This structure is described as a three-way junction. The first oligonucleotide and the second oligonucleotide are target-specific.

The hybridisation of the complementary regions forms a double-stranded initiation site at which the polymerase can bind. The portion of the first oligonucleotide that is complementary to the second oligonucleotide has a 3′ end. The polymerase extends the 3′ end of the first oligonucleotide in a nucleic acid synthesis reaction in the 5′ to 3′ direction, using the second oligonucleotide as the template strand. Polymerisation involves the incorporation of additional free nucleotides. The product of this synthesis is an extended first oligonucleotide, i.e. a nucleic acid comprising the first oligonucleotide and a newly synthesised portion that is complementary to the template strand. The extended first oligonucleotide may be temporally double stranded.

The third oligonucleotide hybridises to the newly synthesised portion of the extended first oligonucleotide at a complementary region. The hybridisation of the complementary regions forms a double-stranded initiation site at which the polymerase can bind. The third oligonucleotide has a 3′ end. The polymerase extends the 3′ end of the third oligonucleotide in a nucleic acid synthesis reaction in the 5′ to 3′ direction, using the extended first oligonucleotide as the template strand. Polymerisation involves the incorporation of additional free nucleotides. The product of this synthesis is an extended third oligonucleotide which results in a double stranded nucleic acid. This double stranded nucleic acid is formed of the extended first oligonucleotide as one strand and the extended third oligonucleotide as the other strand. The double stranded nucleic acid may be duplex DNA.

In some aspects, the method does not involve the transcription of RNA. The method does not cause the transcription of RNA containing at least one active promoter.

Hybridisation of the oligonucleotides and polymerisation may occur at the same temperature. Alternatively, hybridisation of the oligonucleotides and polymerisation may occur at different temperatures. Hybridisation of the oligonucleotides may occur at multiple temperatures or over a temperature gradient. In some aspects, the method may be performed in a single step. Alternatively, the method may be performed in a plurality of steps.

The method may be multiplexed to detect several double stranded nucleic acids simultaneously. Multiplexing may be achieved by any means known to one skilled in the art. For example, multiplexing may involve multiple target molecules and/or multiple sets of oligonucleotides with different target sequence specificities. Detection of the various double stranded nucleic acids may be achieved by any means known to one skilled in the art. For example, the various double stranded nucleic acids may be detected by differentially labelled probes, such as probes labelled with different fluorescent dyes, and/or by using probes with different melting temperatures.

The double stranded nucleic acid may be detected directly or indirectly by any technique known to one skilled in the art. Such techniques include double stranded DNA binding dyes (for example, SYBR Green I dye), hydrolysis probes, TaqMan probes, molecular beacons, hybridisation probes, dual-hybridisation probes, Scorpions probes, gel electrophoresis (with or without the incorporation of labelled bases during the synthesis) and solid surface capture. The detection step may detect a single strand of the double stranded nucleic acid or both strands of the double stranded nucleic acid. Preferably, the detection step comprises detecting the extended first nucleotide, and more preferably, the newly synthesised portion of the extended first nucleotide.

The double stranded nucleic acid may be detected using a probe. The probe may be a universal probe (i.e. the probe is not target-specific) or a non-universal probe. Preferably, the probe is complementary to the newly synthesised portion of the extended first oligonucleotide. For example, the probe may be complementary to a sequence within the newly synthesised portion of the extended first oligonucleotide.

Alternatively, the probe may be complementary to the first oligonucleotide. For example, the probe may be complementary to a sequence within the first portion of the first oligonucleotide, or a sequence within the second portion of the first oligonucleotide, or a sequence within the first and second portions of the first oligonucleotide. Alternatively, the probe may be complementary to a sequence within the first oligonucleotide and the newly synthesised portion.

The nucleic acid synthesised in the method may be amplified prior to detection. Any amplification method known to one skilled in the art may be used. Such methods include polymerase chain reaction (PCR), quantitative PCR and isothermal amplification (for example, loop-mediated isothermal amplification (LAMP), strand displacement amplification (SDA), helicase-dependent amplification (HDA) and nicking enzyme amplification reaction (NEAR). Preferably, the amplification method used is PCR. Any variant of PCR may be used. For example, asymmetric PCR, quantitative PCR, Hot-start PCR, touchdown PCR, methylation-specific PCR, multiplex PCR or nested PCR may be used.

It will be appreciated by one skilled in the art that when terminology is used such as “hybridises . . . to a portion” and “detecting . . . a portion”, the entire portion is not necessarily hybridised or detected. A region within the portion may be hybridised or detected.

In a second embodiment, the present invention provides a method for detecting the presence of a nucleic acid target sequence, the method comprising the steps of:
a) adding a first, a second, a third and a fourth oligonucleotide to a sample comprising nucleic acid sequences, wherein:
   i. the first oligonucleotide comprises a first portion and a second portion, the first portion being complementary to a first portion of the second oligonucleotide and the second portion being complementary to a first portion of either the target sequence or the fourth oligonucleotide;
   ii. the second oligonucleotide comprises a first portion and a second portion, the first portion being complementary to the first portion of the first oligonucleotide and the second portion being complementary to a first portion of either the fourth oligonucleotide or the target sequence;
   iii. the fourth oligonucleotide comprises a first portion and a second portion, the first portion being complementary to the second portion of either the first or second oligonucleotide and the second portion being complementary to a second portion of the target sequence;
b) adding a polymerase to the sample, wherein when the target sequence is present in the sample:
   i. the polymerase initiates nucleic acid synthesis from the first oligonucleotide using the second oligonucleotide as a template strand to generate an extended first oligonucleotide;
   ii. the third oligonucleotide comprises a portion that is complementary and hybridises to the newly synthesised portion of the extended first oligonucleotide; and
   iii. the polymerase initiates nucleic acid synthesis from the third oligonucleotide using the extended first oligonucleotide as a template strand to generate a double stranded nucleic acid;
c) detecting the double stranded nucleic acid.

The first oligonucleotide, the second oligonucleotide, the target sequence and the fourth oligonucleotide hybridise to each other at their respective complementary regions to form a four strand structure. This structure is described as a four-way junction.

In one orientation of the four-way junction, the second oligonucleotide and the fourth oligonucleotide are target-specific and the first oligonucleotide is universal. A target-specific oligonucleotide is designed to hybridise to a specific target sequence, whereas a universal oligonucleotide is not designed to hybridise to a specific target sequence. In this orientation, the second portion of the first oligonucleotide is complementary to the first portion of the fourth oligonucleotide, and the second portion of the second oligonucleotide is complementary to the first portion of the target sequence (and for the avoidance of doubt, the first portion of the fourth oligonucleotide is complementary to the second portion of the first oligonucleotide).

In a second orientation of the four-way junction, the first oligonucleotide and the fourth oligonucleotide are target-specific and the second oligonucleotide is universal. In this orientation, the second portion of the first oligonucleotide is complementary to the first portion of the target sequence, and the second portion of the second oligonucleotide is complementary to the first portion of the fourth oligonucleotide (and for the avoidance of doubt, the first portion of the fourth oligonucleotide is complementary to the second portion of the second oligonucleotide).

The way in which this second embodiment of the detection method functions is very similar to the way in which the first embodiment function. Therefore, much of the description above for the first embodiment is equally applicable to this second embodiment. For example, amongst other parts, the description of the target sequence, the polymerase, the binding of the polymerase to the double stranded initiation site formed by the first and second oligonucleotides, the extension of the first oligonucleotide, binding and extension of the third oligonucleotide, and detection and amplification of the double stranded nucleic acid for the first embodiment also applies to the second embodiment.

The first, second, third and fourth oligonucleotides may be formed from DNA, peptide nucleic acids (PNA), locked nucleic acids (LNA) or any combination thereof. Preferably, the oligonucleotides are formed from DNA. In some embodiments, the first oligonucleotide is formed from DNA, the second oligonucleotide is formed from DNA, PNA, LNA or any combination thereof, the third oligonucleotide is formed from DNA, and the fourth oligonucleotide is formed from DNA, PNA, LNA or any combination thereof.

The oligonucleotides may be any length of nucleotides (nt) which allow them to effectively hybridise to their respective complementary sequences.

### First Oligonucleotide

The first oligonucleotide may be 10-80 nt in length. In some embodiments, the first oligonucleotide is 15-70 nt. In various embodiments, the first oligonucleotide is 20-60 nt. In a number of embodiments, the first oligonucleotide is 25-55 nt. In certain embodiments, the first oligonucleotide is 30-50 nt. In particular embodiments, the first oligonucleotide is 35-45 nt.

The first oligonucleotide comprises a first portion and a second portion, the first portion being complementary to a first portion of the second oligonucleotide and the second portion being complementary to a first portion of either the target sequence or the fourth oligonucleotide.

The first portion of the first oligonucleotide may be 5-40 nt in length. In some embodiments, the first portion of the first oligonucleotide is 10-35 nt. In various embodiments, the first portion of the first oligonucleotide is 15-30 nt. In a number of embodiments, the first portion of the first oligonucleotide is 15-25 nt.

The second portion of the first oligonucleotide may be 5-40 nt in length. In some embodiments, the second portion of the first oligonucleotide is 10-35 nt. In various embodiments, the second portion of the first oligonucleotide is 15-30 nt. In a number of embodiments, the second portion of the first oligonucleotide is 15-25 nt.

In some embodiments, the first portion of the first oligonucleotide is positioned on the 3′ side of the second portion. In various embodiments, the first portion is positioned at the 3′ end of the first oligonucleotide.

In a number of embodiments, the first portion and second portion of the first oligonucleotide are separated by 0-10 nucleotides. In particular embodiments, the first portion and second portion are separated by 0-5 nucleotides. In certain embodiments, the first portion and second portion are separated by 0-2 nucleotides. In some embodiments, the first portion and second portion are separated by 0 nucleotides, i.e. they are contiguous.

### Second Oligonucleotide

The second oligonucleotide may be 50-250 nt in length. In some embodiments, the second oligonucleotide is 70-200 nt. In various embodiments, the second oligonucleotide is 90-180 nt. In a number of embodiments, the second oligonucleotide is 100-170 nt. In certain embodiments, the second oligonucleotide is 110-160 nt. In particular embodiments, the second oligonucleotide is 120-150 nt.

The second oligonucleotide comprises a first portion and a second portion, the first portion being complementary to the first portion of the first oligonucleotide and the second portion being complementary to a first portion of either the fourth oligonucleotide or the target sequence.

The second oligonucleotide comprises a third portion that is homologous to the third oligonucleotide. When the first oligonucleotide is extended using the second oligonucleotide as the template strand, the complement to the third oligonucleotide is synthesised.

The first portion of the second oligonucleotide may be 5-40 nt in length. In some embodiments, the first portion of the second oligonucleotide is 10-35 nt. In various embodiments, the first portion of the second oligonucleotide is 15-30 nt. In a number of embodiments, the first portion of the second oligonucleotide is 15-25 nt.

The second portion of the second oligonucleotide may be 5-40 nt in length. In some embodiments, the second portion of the second oligonucleotide is 10-35 nt. In various embodiments, the second portion of the second oligonucleotide is 15-30 nt. In a number of embodiments, the second portion of the second oligonucleotide is 15-25 nt.

In some embodiments, the first portion of the second oligonucleotide is positioned on the 5′ side of the second portion. In various embodiments, the second portion is the closest portion to the 3′ end of the second oligonucleotide. In certain embodiments, the second portion is positioned at the 3′ end of the second oligonucleotide.

In a number of embodiments, the first portion and second portion of the second oligonucleotide are separated by 0-10 nucleotides. In particular embodiments, the first portion and second portion are separated by 0-5 nucleotides. In certain embodiments, the first portion and second portion are separated by 0-2 nucleotides. In some embodiments, the first portion and second portion are separated by 0 nucleotides, i.e. they are contiguous.

The second oligonucleotide is used as a template strand when the first oligonucleotide is extended by the polymerase. The portion of the second oligonucleotide that is used as a template strand may be 50-200 nt. In some embodiments, this portion is 60-140 nt. In various embodiments, this portion is 70-120 nt. In a number of embodiments, this portion is 80-100 nt.

In various embodiments, the portion of the second oligonucleotide that is used as a template strand is positioned on the 5′ side of the first portion. This portion may comprise the third portion that is homologous to the third oligonucleotide.

### Third Oligonucleotide

The third oligonucleotide may be 10-50 nt in length. In a number of embodiments, the third oligonucleotide is 10-45 nt. In certain embodiments, the third oligonucleotide is 15-40 nt. In particular embodiments, the third oligonucleotide is 20-35 nt.

The third oligonucleotide comprises a portion that is complementary and hybridises to the newly synthesised portion of the extended first oligonucleotide. Put another way, the third oligonucleotide comprises a portion that is homologous to a third portion of the second oligonucleotide (which is used as a template strand when the first oligonucleotide is extended).

### Fourth Oligonucleotide

The fourth oligonucleotide may be 10-80 nt in length. In some embodiments, the fourth oligonucleotide is 15-70 nt. In various embodiments, the fourth oligonucleotide is 20-60 nt. In a number of embodiments, the fourth oligonucleotide is 25-55 nt. In certain embodiments, the fourth oligonucleotide is 30-50 nt. In particular embodiments, the fourth oligonucleotide is 35-45 nt.

The fourth oligonucleotide comprises a first portion and a second portion, the first portion being complementary to the second portion of either the first or second oligonucleotide and the second portion being complementary to a second portion of the target sequence.

The first portion of the fourth oligonucleotide may be 5-40 nt in length. In some embodiments, the first portion of the fourth oligonucleotide is 10-35 nt. In various embodiments, the first portion of the fourth oligonucleotide is 15-30 nt. In a number of embodiments, the first portion of the fourth oligonucleotide is 15-25 nt.

The second portion of the fourth oligonucleotide may be 5-40 nt in length. In some embodiments, the second portion of the fourth oligonucleotide is 10-35 nt. In various embodiments, the second portion of the fourth oligonucleotide is 15-30 nt. In a number of embodiments, the second portion of the fourth oligonucleotide is 15-25 nt.

Depending on the orientation of the four-way junction formed by the oligonucleotides, the first portion of the fourth oligonucleotide may be positioned on the 3′ side of the second portion. Alternatively, the first portion of the fourth oligonucleotide may be positioned on the 5′ side of the second portion.

In a number of embodiments, the first portion and second portion of the fourth oligonucleotide are separated by 0-10 nucleotides. In particular embodiments, the first portion and second portion are separated by 0-5 nucleotides. In certain embodiments, the first portion and second portion are separated by 0-2 nucleotides. In some embodiments, the first portion and second portion are separated by 0 nucleotides, i.e. they are contiguous.

The fourth oligonucleotide may be synthesised using any method known to one skilled in the art. The fourth oligonucleotide may be composed of entirely canonical nucleobases. Alternatively, the fourth oligonucleotide may comprise canonical and non-canonical nucleobases. The fourth oligonucleotide may be unmodified.

Alternatively, the fourth oligonucleotide may be modified. The fourth oligonucleotide may be modified by any technique known to one skilled in the art. Such techniques include modifying the DNA backbone, e.g. bridged nucleic acids, methylphosphonate backbone, phosphodiester (PO) backbone, phosphorothioate (PS) backbone and phosphorodithioate (PdiS) backbone. Such techniques also include modifying the bases, e.g. terminator bases, dideoxynucleotides, 2-aminopurine, 2,6-diaminopurine (2-amino-dA), 5-bromo-deoxyuridine, deoxyuridine, inverted dT, inverted dideoxy-T, dideoxycytidine, 5-methyl deoxycytidine, deoxyinosine, 5-hydroxybutynl-2′-deoxyuridine, 8-aza-7-deazaguanosine, locked nucleic acids (LNAs), peptide nucleic acids (PNAs), 5-nitroindole, 2′-O-methyl RNA bases, hydroxymethyl dC, iso-dC, iso-dG, fluoro-bases, fluorescently-labelled bases and 2′-O-methoxy-ethyl bases (2′-MOE). Preferably, the fourth oligonucleotide is modified. More preferably, the fourth oligonucleotide is modified such that it cannot form an initiation site for a polymerase. Even more preferably, the fourth oligonucleotide is modified using a chain terminator, such as a dideoxynucleotide.

In both embodiments described above (i.e. the three-way junction system and the four-way junction system), the junction forms in the presence of the target sequence but does not form in the absence of the target sequence. The junction forms in the presence of a suitable concentration of the target sequence at a suitable annealing temperature.

In the presence of the target sequence, the melting temperature of the complex may be above the annealing temperature such that the complex is stable at the annealing temperature of the method. When the complex is stable, polymerisation may proceed.

In the absence of the target sequence, the melting temperature of the complex may be equal to or below the annealing temperature such that the complex is not stable at the annealing temperature of the method. When the complex is not stable, polymerisation may not proceed.

One skilled in the art will be able to calculate the optimal annealing temperature for the oligonucleotides provided in the method. The annealing temperature may be between 40-70° C., preferably between 50-65° C., more preferably between 55-60° C. In one aspect, polymerisation may proceed at a temperature between 45-80° C., preferably between 50-75° C., more preferably between 55-70° C., even more preferably between 60-65° C. Polymerisation may proceed when the complex is formed in the presence of the target sequence.

The annealing temperature is the temperature at which annealing (i.e. hybridisation of the oligonucleotides and the target sequence) and optionally extension of oligonucleotides occurs. The annealing temperature relies directly on length and composition of the oligonucleotides. The optimal annealing temperature (TₐOpt) for a given primer pair on a particular target can be calculated using the following equation:

TₐOpt=0.3×(Tₘ of primer)+0.7×(Tₘ of product)−14.9

where
Tₘ of primer is the melting temperature of the less stable primer-template pair
Tₘ of product is the melting temperature of the PCR product

The melting temperature (Tₘ) of an oligonucleotide is the temperature at which 50% of the oligonucleotide is duplexed with its perfect complement and 50% is free in solution. Tₘ can be determined experimentally by measuring the absorbance change of the oligonucleotide with its complement as a function of temperature. For example, absorbance measurements at 260 nm can be made in a thermostated cell in a UV-Vis spectrophotometer. The Tₘ is the reading halfway between the double-stranded DNA and single-stranded DNA plateaus. The melting temperature can also be determined theoretically by methods known to one skilled in the art. Such methods include the Nearest Neighbours method and the basic method.

The present invention provides the advantage that the double stranded nucleic acid is not generated (and therefore not detected) when the target nucleic acid is not present. This invention is therefore suitable for the detection of a target nucleic acid which can be monitored in real time using a detection system. Furthermore, this technology has the advantage that reverse transcriptase is not required for the detection of an RNA target molecule. In both embodiments, the third oligonucleotide may be universal and not specific to any target. The utilisation of the four-way junction system further allows one of the first and second oligonucleotides to be universal and not specific to any target.

The present invention also provides a first, second, third and/or fourth oligonucleotide as described herein for use in the methods described herein.

Further, the present invention provides the use of a first, second, third and/or fourth oligonucleotide as described herein in the methods described herein.

The present invention also provides a kit for detecting the presence of a nucleic acid target sequence, the kit comprising the first, second and third oligonucleotides described herein. The kit may optionally include the fourth oligonucleotide described herein.

### BRIEF DESCRIPTION OF THE DRAWINGS

The invention will now be described in detail by way of example only with reference to the figures in which:

FIG. 1 shows a three-way junction system.

FIG. 2 shows an exemplary step-by-step process used to detect a target nucleic acid.

FIG. 3A shows the first of the four-way junction systems.

FIG. 3B shows the second of the four-way junction systems.

FIG. 4A shows the formation of the junction using the oligonucleotides detailed in Table 1 of the example.

FIG. 4B shows the amplification curves generated in the example.

### DETAILED DESCRIPTION OF THE INVENTION

The invention disclosed herein enables the detection of RNA in a sample without the need for a reverse transcription enzyme or cDNA transcription step. The invention is effected by use of a DNA/RNA junction with three or four branches or strands, where the target ribonucleic acid is an essential component without which the junction cannot be stably formed, at a suitable concentration and time for the step to occur, at the Tₐ (annealing temperature—the temperature at which annealing and optionally extension of oligonucleotides occurs) of the reaction. The invention further comprises, as one element of the junction, a DNA polymer, approximately 60-200 bases in length comprising a ‘junction-forming’ portion, which is optionally target-specific, a universal sequence homologous to that of a DNA probe and a sequence that is complementary to a universal primer. This universal sequence element further allows use of a universal fluorescent probe, thus further reducing the cost of assay development. Multiplexing may be achieved by use of multiple universal target, primer and probe sequences. In some examples of the invention, it may be possible to multiplex by single primer sequence only, thus allowing sharing of probe and reverse primer between common targets. This is particularly relevant when used in combination with hybridisation probes and melting curve analysis.

The removal of the need for a reverse transcription step in addition to use of a universal detection method compatible with multiple target assays has the potential to substantially simplify RNA detection in terms of both design and reaction composition, since it enables use of an off-the-shelf qPCR master mix and can use conserved oligonucleotide regions and probes.

The invention disclosed herein enables the indirect specific detection of an RNA target through the amplification of a universal synthetic DNA element. The invention comprises the following components:
1) A ‘forward junction primer’ (FJP—the first oligonucleotide described above) comprising two elements, which, when both elements are hybridised to an appropriate nucleic acid has a Tm such that, under the conditions of the reaction, it is able to extend to form a complementary strand to the ‘universal target element’ (UTE).
2) A ‘universal target element’ (UTE—the second oligonucleotide described above) comprising, at its 5′ end, a universal sequence homologous to a ‘universal reverse primer’ (URP). The sequence further comprises a universal probe-specific sequence homologous to that of the ‘universal probe’ (UP) such that, when its complement is generated, the universal probe is able to hybridise. The sequence further comprises a target sequence to which the 3′ end of the FJP is able to hybridise when a junction is formed. The sequence further comprises a 3′ junction forming element that enables the UTE to hybridise to an additional nucleotide sequence. This 3′-sequence may be universal or may be a target-specific element.
3) A ‘universal reverse primer’ (URP—the third oligonucleotide described above), having a Tm such that, under amplification conditions provided, it is able to hybridise to the complementary strand to the UTE (i.e. the extended FJP) and be extended.
4) Optionally a universal probe homologous to a portion of the UTE to enable quantitative real-time detection. The universal probe is optionally labelled, for example with a fluorophore or for electrochemical detection, and is optionally a hydrolysis probe.
5) Optionally an additional (non-extending) blocked oligonucleotide (ABO—the fourth oligonucleotide described above) sequence that, when incorporated into the reaction under amplification conditions provided, is able to stabilise a four-way junction, said junction to be formed from the FJP, the UTE, the ABO and the specific target RNA.
6) Optionally, a label may be incorporated into the FJP as an alternative probing method.

The components above are combined in a manner such that the universal element is amplified in the presence of the target nucleic acid but not in its absence under the amplification conditions provided. The reaction occurs in the manner detailed in the drawings and descriptions below.

The reaction as described may be performed as a standard qPCR reaction in an off-the-shelf hydrolysis probe reaction mix. Example reagent concentrations are also described.

This does not limit its applications to those explicitly described, nor does it limit the scope of the invention. Elements of the invention, for example the FJP and UTE may equally be incorporated into an isothermal amplification reaction. The invention may be improved by further modification of the oligonucleotides, for example by use of modified bases, LNA, PNA, etc. Alternative probe types may be used, for example hybridisation probes. Detection is not explicitly limited to fluorescence, and this method may be incorporated into alternative systems such as those performing detection by electrochemistry and hydrogen ion release. Similarly, this invention is not limited to the reaction mixtures and cycling conditions described. The invention may be improved by modification of salt and buffer conditions, addition of crowding agents, and use of additives capable of modifying the melting temperature of nucleic acids.

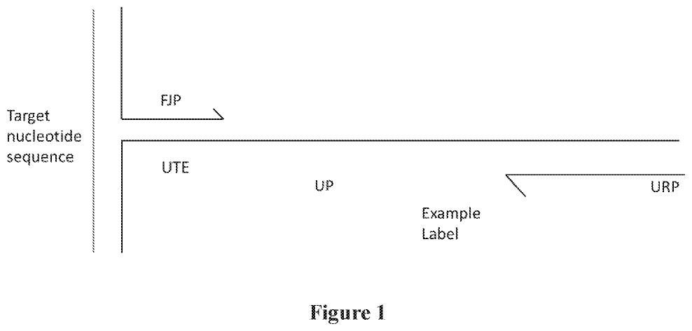

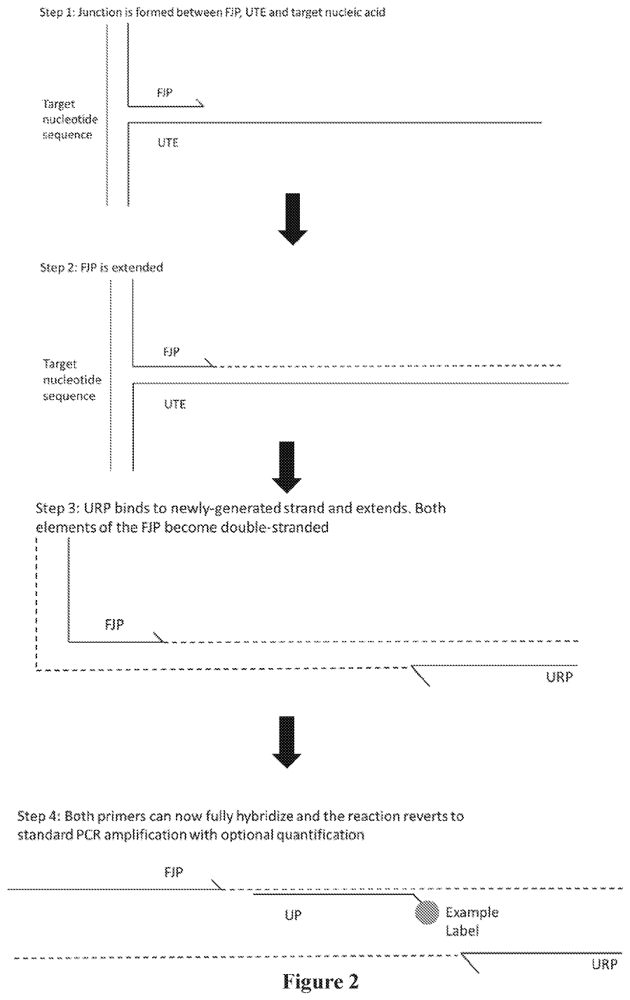

FIG. 1 shows a three-way junction version of the junction system. The 3′-element of the FJP oligonucleotide is able to hybridise to its complementary portion of the UTE. In the absence of a target nucleotide sequence the Tₘ (melting temperature) of this interaction is insufficient to produce stable hybridisation at the Tₐ (annealing temperature) of the amplification reaction and the FJP is not extended. In the presence of the target nucleotide sequence, both the 5′-element of the FJP and the 3′-element of the UTE are able to hybridise to the target nucleotide and the junction becomes stable at the Tₐ of the reaction. Once the junction is stable, the polymerase chain reaction is able to proceed in the following steps, as detailed in FIG. 2:
1) The FJP is extended along the UTE to generate a double-stranded universal target sequence with a mismatched tail at its 5′-end.
2) The URP hybridises to the sequence generated and is extended through the universal sequences to generate a double-stranded target sequence incorporating the entire FJP sequence.
3) From this point the amplicon generated performs as a standard PCR target and can be amplified independently of the RNA target. Detection may be performed by a standard hydrolysis or hybridisation probe.

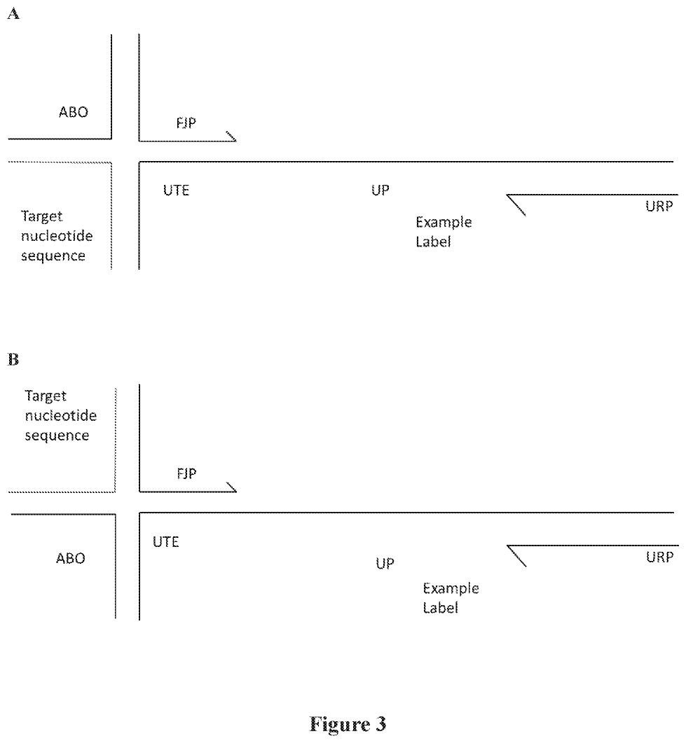

FIG. 3A shows the first of the four-way junction systems. The 3′-element of the FJP oligonucleotide is able to hybridise to its complementary portion of the UTE while the 5′-element is able to hybridise to the 3′-element of the ABO. In the absence of a target nucleotide sequence the Tₘ (melting temperature) of the interaction between the ABO and the FJP is insufficient to produce stable hybridisation at the Tₐ (annealing temperature) of the amplification reaction and the FJP is not extended. In the presence of the target nucleotide sequence, both the 5′-element of the ABO and the 3′-element of the UTE are able to hybridise to the target nucleotide and the four-way junction becomes stable at the Tₐ of the reaction. Once the junction is stable, the polymerase chain reaction is able to proceed in the following steps:
1) The FJP is extended along the UTE to generate a double-stranded universal target sequence with a mismatched tail at its 5′-end.
2) The URP hybridises to the sequence generated and is extended through the universal sequences to generate a double-stranded target sequence incorporating the entire FJP sequence.
3) From this point the amplicon generated performs as a standard PCR target and can be amplified independently of the RNA target. Detection may be performed by a standard hydrolysis or hybridisation probe.

FIG. 3B shows the second of the four-way junction systems. The 3′-element of the FJP oligonucleotide is able to hybridise to its complementary portion of the UTE. In the absence of a target nucleotide sequence the Tₘ (melting temperature) of the interaction between the FJP and the UTE is insufficient to produce stable hybridisation at the Tₐ (annealing temperature) of the amplification reaction and the FJP is not extended. In the presence of the target nucleotide sequence, the target sequence is hybridised to the 3′-element of the ABO and the 5′-element of the FJP. The 5′ element of the ABO is also able to hybridise to the 3′-element of the UTE and the four-way junction becomes stable at the Tₐ of the reaction. Once the junction is stable, the polymerase chain reaction is able to proceed in the following steps:
1) The FJP is extended along the UTE to generate a double-stranded universal target sequence with a mismatched tail at its 5′-end.
2) The URP hybridises to the sequence generated and is extended through the universal sequences to generate a double-stranded target sequence incorporating the entire FJP sequence.

From this point the amplicon generated performs as a standard PCR target and can be amplified independently of the RNA target. Detection may be performed by a standard hydrolysis or hybridisation probe.

### EXAMPLES

This example details one example of the application of the three-way junction system.

Promega GoTaq probe qPCR master mix (Promega, UK) was combined with the components below to produce a reaction mixture comprising 1× concentration Promega GoTaq master mix and the oligonucleotides at the concentrations described. Note that the FluB_target is an RNA oligonucleotide.

TABLE 1

| | | Concentration/ |
|---|---|---|
| Oligo ID | Oligo Sequence | reaction |
| FluB_FJP | AGACTCCCACCGCAGTTTCTTTGGTCTGGCTGT | 900 nM |
| | GATCTAGA (SEQ ID NO. 1) | |
| FluB_UTE | GTCTGATCAACCTTCAAACAGATCTAGAGTCT | 50 nM |
| | AAAACAGTGATCTCCTGCGTGCGAGATAGAA | |
| | ATACTAGGTAACTACAGGGACTGCGACGTTCT | |
| | AGATCACAGCCAGACCAAAAGCTGCTCGAAT | |
| | TGGCTTTG (SEQ ID NO. 2) | |
| FluB_URP | CAGATCTAGAGTCTAAAACAGTGATCTCCTG | 900 nM |
| | CG (SEQ ID NO. 3) | |
| FluB_UP | /56-FAM/CGAGATAGAAATACTAGGTAACTAC | 300 nM |
| | AGGGACTGC/36-TAMSp/ (SEQ ID NO. 4) | |
| FluB_target | CUGCAAAGCCAAUUCGAGCAGCUGAAACUG | 8 pM-8 nM |
| | CGGUGGGAGUCUCUG (RNA) (SEQ ID NO. 5) | |

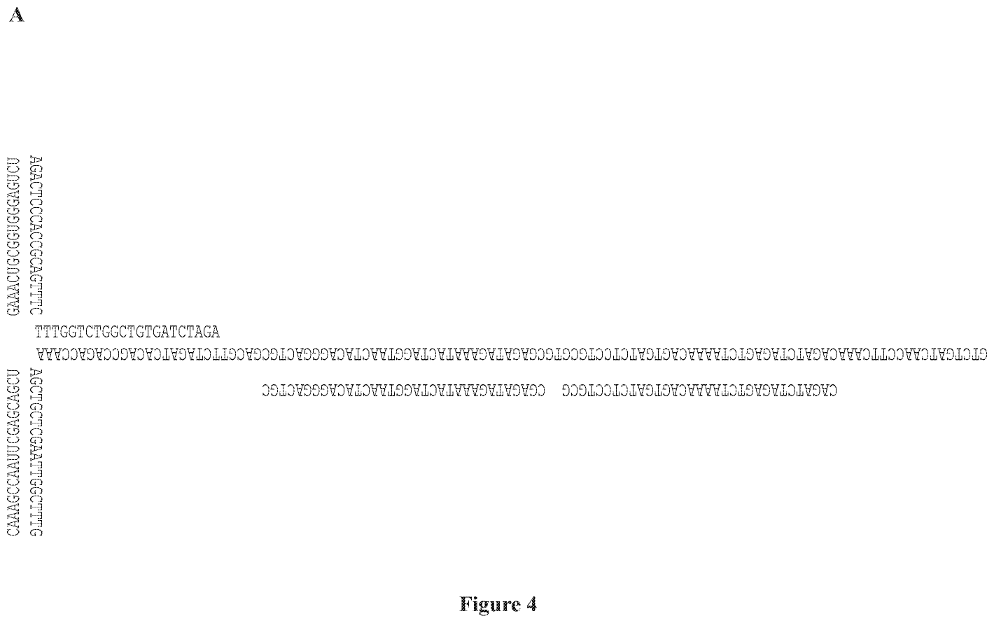

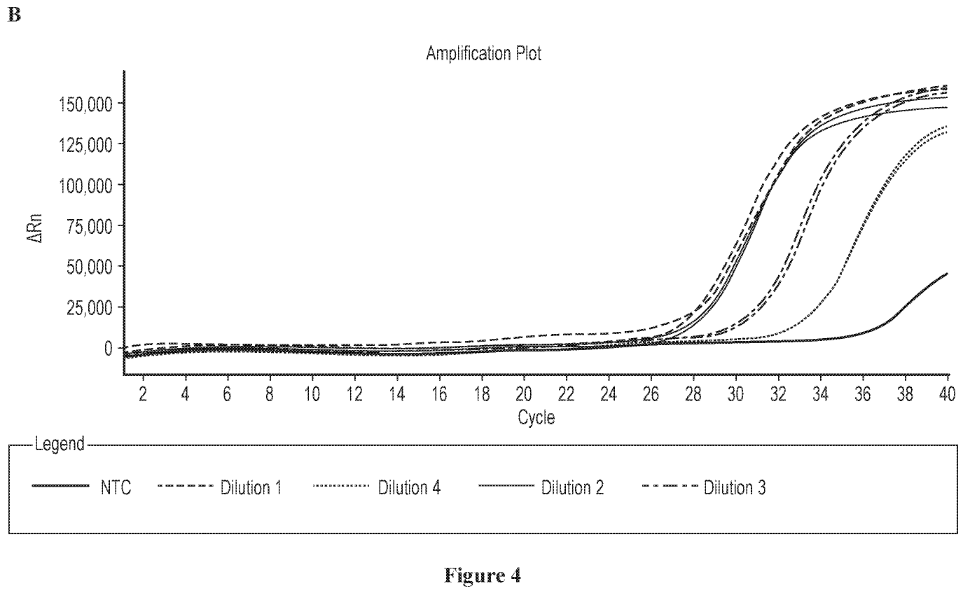

FIG. 4A shows the formation of the three-way junction using the oligonucleotides detailed Table 1.

The Promega GoTaq polymerase was activated using a two-minute hot start at 95° C. This was followed by forty amplification cycles comprising a 5 second hold at 95° C. followed by a 30 second hold at 60° C. with fluorescent detection. Amplification curves are depicted in FIG. 4B and Cₜ values are shown in Table 2 below.

TABLE 2

| Concentration of FluB_target | Cₜ Mean |
|---|---|
| 8 nM | 28.2 |
| 800 pM | 28.7 |
| 80 pM | 31.0 |
| 8 pM | 33.9 |
| NTC | 37.9 |

The amplification data clearly shows a concentration-dependent amplification effect. At high concentrations, Cₜ values are determined by the ability of the nucleotides to form a junction, while below this, Cₜ is limited by target input concentration. Sensitivity and specificity may be further improved by modifications to reaction mixes, amplification conditions and oligonucleotide concentrations.

1. A method of detecting the presence of a nucleic acid target sequence, the method comprising the steps of:
a) adding a first, a second and a third oligonucleotide to a sample comprising nucleic acid sequences, wherein:
i. the first oligonucleotide comprises a first portion and a second portion, the first portion being complementary to a first portion of the second oligonucleotide and the second portion being complementary to a first portion of the target sequence;
ii. the second oligonucleotide comprises a first portion and a second portion, the first portion being complementary to the first portion of the first oligonucleotide and the second portion being complementary to a second portion of the target sequence;
b) adding a polymerase to the sample, wherein when the target sequence is present in the sample:
i. the polymerase initiates nucleic acid synthesis from the first oligonucleotide using the second oligonucleotide as a template strand to generate an extended first oligonucleotide;
ii. the third oligonucleotide comprises a portion that is complementary and hybridises to the newly synthesised portion of the extended first oligonucleotide; and
iii. the polymerase initiates nucleic acid synthesis from the third oligonucleotide using the extended first oligonucleotide as a template strand to generate a double stranded nucleic acid;
c) detecting the double stranded nucleic acid.

2. A method of detecting the presence of a nucleic acid target sequence, the method comprising the steps of:
a) adding a first, a second, a third and a fourth oligonucleotide to a sample comprising nucleic acid sequences, wherein:
i. the first oligonucleotide comprises a first portion and a second portion, the first portion being complementary to a first portion of the second oligonucleotide and the second portion being complementary to a first portion of either the target sequence or the fourth oligonucleotide;
ii. the second oligonucleotide comprises a first portion and a second portion, the first portion being complementary to the first portion of the first oligonucleotide and the second portion being complementary to a first portion of either the fourth oligonucleotide or the target sequence;
iii. the fourth oligonucleotide comprises a first portion and a second portion, the first portion being complementary to the second portion of either the first or second oligonucleotide and the second portion being complementary to a second portion of the target sequence;
b) adding a polymerase to the sample, wherein when the target sequence is present in the sample:
i. the polymerase initiates nucleic acid synthesis from the first oligonucleotide using the second oligonucleotide as a template strand to generate an extended first oligonucleotide;
ii. the third oligonucleotide comprises a portion that is complementary and hybridises to the newly synthesised portion of the extended first oligonucleotide; and
iii. the polymerase initiates nucleic acid synthesis from the third oligonucleotide using the extended first oligonucleotide as a template strand to generate a double stranded nucleic acid;
c) detecting the double stranded nucleic acid.

3. The method according to claim 1, wherein the nucleic acid target sequence is an RNA target sequence.

4. The method according to claim 1, wherein the polymerase added to the sample is a DNA polymerase.

5. The method according to claim 1, wherein the double stranded nucleic acid is subjected to an amplification step prior to the detection step.

6. The method according to claim 5, wherein the amplification step is performed using polymerase chain reaction.

7. The method according to claim 2, wherein the second portion of the first oligonucleotide is complementary to the first portion of the fourth oligonucleotide, the second portion of the second oligonucleotide is complementary to the first portion of the target sequence, and the first portion of the fourth oligonucleotide is complementary to the second portion of the first oligonucleotide.

8. The method according to claim 2, wherein the second portion of the first oligonucleotide is complementary to the first portion of the target sequence, the second portion of the second oligonucleotide is complementary to the first portion of the fourth oligonucleotide, and the first portion of the fourth oligonucleotide is complementary to the second portion of the second oligonucleotide.

9. The method according to claim 1 comprising the steps of:
a) adding a first, a second and a third oligonucleotide to a sample comprising RNA nucleic acid sequences, wherein:
i. the first oligonucleotide comprises a first portion and a second portion, the first portion being complementary to a first portion of the second oligonucleotide and the second portion being complementary to a first portion of the target sequence, wherein the first portion is positioned at the 3′ end of the first oligonucleotide and the second portion is on the 5′ side of the first portion;
ii. the second oligonucleotide comprises a first portion, a second portion and a third portion, the first portion being complementary to the first portion of the first oligonucleotide, the second portion being complementary to a second portion of the target sequence, and the third portion being homologous to a portion of the third oligonucleotide, wherein the first portion is positioned on the 5′ side of the second portion and the third portion is positioned on the 5′ side of the first portion;
b) adding a DNA polymerase to the sample, wherein when the RNA target sequence is present in the sample:
i. the polymerase initiates nucleic acid synthesis from the 3′ end of the first oligonucleotide using the second oligonucleotide as a template strand to generate an extended first oligonucleotide;
ii. the third oligonucleotide comprises a portion that is complementary and hybridises to the newly synthesised portion of the extended first oligonucleotide; and
iii. the DNA polymerase initiates nucleic acid synthesis from the 3′ end of the third oligonucleotide using the extended first oligonucleotide as a template strand to generate a double stranded nucleic acid;
c) amplifying the double stranded nucleic acid using a polymerase chain reaction; and
d) detecting the double stranded nucleic acid.

10. The method according to claim 2 comprising the steps of:
a) adding a first, a second, a third and a fourth oligonucleotide to a sample comprising RNA nucleic acid sequences, wherein:
i. the first oligonucleotide comprises a first portion and a second portion, the first portion being complementary to a first portion of the second oligonucleotide and the second portion being complementary to a first portion of either the target sequence or the fourth oligonucleotide, wherein the first portion is positioned at the 3′ end of the first oligonucleotide and the second portion is on the 5′ side of the first portion;
ii. the second oligonucleotide comprises a first portion, a second portion and a third portion, the first portion being complementary to the first portion of the first oligonucleotide, the second portion being complementary to a first portion of either the fourth oligonucleotide or the target sequence, and the third portion being homologous to a portion of the third oligonucleotide, wherein the first portion is positioned on the 5′ side of the second portion and the third portion is positioned on the 5′ side of the first portion;
iii. the fourth oligonucleotide comprises a first portion and a second portion, the first portion being complementary to the second portion of either the first or second oligonucleotide and the second portion being complementary to a second portion of the target sequence;
b) adding a DNA polymerase to the sample, wherein when the RNA target sequence is present in the sample:
i. the polymerase initiates nucleic acid synthesis from the 3′ end of the first oligonucleotide using the second oligonucleotide as a template strand to generate an extended first oligonucleotide;
ii. the third oligonucleotide comprises a portion that is complementary and hybridises to the newly synthesised portion of the extended first oligonucleotide; and
iii. the DNA polymerase initiates nucleic acid synthesis from the 3′ end of the third oligonucleotide using the extended first oligonucleotide as a template strand to generate a double stranded nucleic acid;
c) amplifying the double stranded nucleic acid using a polymerase chain reaction; and
d) detecting the double stranded nucleic acid.

11. A first, second or third oligonucleotide for use in the method of claim 1, wherein:
a) the first oligonucleotide comprises a first portion and a second portion, the first portion being complementary to a first portion of the second oligonucleotide and the second portion being complementary to a first portion of the target sequence;
b) the second oligonucleotide comprises a first portion, a second portion and a third portion, the first portion being complementary to the first portion of the first oligonucleotide, the second portion being complementary to a second portion of the target sequence, and the third portion being homologous to a portion of the third oligonucleotide; and/or
c) the third oligonucleotide comprises a portion that is homologous to a third portion of the second oligonucleotide.

12. A first, second, third or fourth oligonucleotide for use in the method of claim 2, wherein:
a) the first oligonucleotide comprises a first portion and a second portion, the first portion being complementary to a first portion of the second oligonucleotide and the second portion being complementary to a first portion of either the target sequence or the fourth oligonucleotide;
b) the second oligonucleotide comprises a first portion, a second portion and a third portion, the first portion being complementary to the first portion of the first oligonucleotide, the second portion being complementary to a first portion of either the fourth oligonucleotide or the target sequence, and the third portion being homologous to a portion of the third oligonucleotide;
c) the third oligonucleotide comprises a portion that is homologous to a third portion of the second oligonucleotide; and/or
d) the fourth oligonucleotide comprises a first portion and a second portion, the first portion being complementary to the second portion of either the first or second oligonucleotide and the second portion being complementary to a second portion of the target sequence.

13. (canceled)

14. (canceled)

15. A kit for detecting the presence of a nucleic acid target sequence, the kit comprising a first, second and third oligonucleotide, wherein:
a) the first oligonucleotide comprises a first portion and a second portion, the first portion being complementary to a first portion of the second oligonucleotide and the second portion being complementary to a first portion of the target sequence;
b) the second oligonucleotide comprises a first portion, a second portion and a third portion, the first portion being complementary to the first portion of the first oligonucleotide, the second portion being complementary to a second portion of the target sequence, and the third portion being homologous to a portion of the third oligonucleotide; and
c) the third oligonucleotide comprises a portion that is homologous to a third portion of the second oligonucleotide.

16. A kit for detecting the presence of a nucleic acid target sequence, the kit comprising a first, second, third and fourth oligonucleotide, wherein:
a) the first oligonucleotide comprises a first portion and a second portion, the first portion being complementary to a first portion of the second oligonucleotide and the second portion being complementary to a first portion of either the target sequence or the fourth oligonucleotide;
b) the second oligonucleotide comprises a first portion, a second portion and a third portion, the first portion being complementary to the first portion of the first oligonucleotide, the second portion being complementary to a first portion of either the fourth oligonucleotide or the target sequence, and the third portion being homologous to a portion of the third oligonucleotide;
c) the third oligonucleotide comprises a portion that is homologous to a third portion of the second oligonucleotide; and
d) the fourth oligonucleotide comprises a first portion and a second portion, the first portion being complementary to the second portion of either the first or second oligonucleotide and the second portion being complementary to a second portion of the target sequence.

17. The method according to claim 2, wherein the nucleic acid target sequence is an RNA target sequence.

18. The method according to 2, wherein the polymerase added to the sample is a DNA polymerase.

19. The method according to 2, wherein the double stranded nucleic acid is subjected to an amplification step prior to the detection step.

20. The method according to claim 19, wherein the amplification step is performed using polymerase chain reaction.


SEQUENCE LISTING (5 sequences)

SEQ ID NO: 1 - DNA, 41 bp, Artificial Sequence
  FluB Forward Junction Primer
agactcccaccgcagtttctttggtctggctgtgatctaga

SEQ ID NO: 2 - DNA, 134 bp, Artificial Sequence
  FluB Universal Target Element
gtctgatcaaccttcaaacagatctagagtctaaaacagtgatctcctgcgtgcgagatagaaatactaggtaactacagggactgcgacgttctagatcacagccagaccaaaagctgctcgaattggctttg

SEQ ID NO: 3 - DNA, 33 bp, Artificial Sequence
  FluB Universal Reverse Primer
cagatctagagtctaaaacagtgatctcctgcg

SEQ ID NO: 4 - DNA, 34 bp, Artificial Sequence
  FluB Universal Probe
cgagatagaaatactaggtaactacagggactgc

SEQ ID NO: 5 - RNA, 45 nt, Artificial Sequence
  FluB Target
cugcaaagccaauucgagcagcugaaacugcggugggagucucug